(19) United States
(12) Patent Application Publication
(10) Pub. No.: US 2003/0153979 A1
(43) Pub. Date: Aug. 14, 2003

(54) Posterior stabilized knee system prosthetic devices employing diffusion-hardened surfaces

(76) Inventors: Dean Hughes, Memphis, TN (US); Harsh Gupta, Memphis, TN (US); Gordon Hunter, Memphis, TN (US)

Correspondence Address: FULBRIGHT & JAWORSKI, LLP, 1301 MCKINNEY, SUITE 5100, HOUSTON, TX, 77010-3095, US

(21) Appl. No.: 10/073,705

(22) Filed: Feb. 11, 2002

(51) Int. Cl. A61F 2/38

(52) U.S. Cl. 623/20.28

(57) ABSTRACT

An orthopedic implant with a diffusion-hardened surface on non-load bearing areas of the implant for interaction with non-load bearing surfaces of a polymeric bio-compatible material, such as UHMWPE. The orthopedic implant is a posterior stabilized knee prosthetic and system where a coating of oxidized zirconium is formed in the cam of the femoral prosthetic for interaction with the central post of a polymeric tibial insert. The diffusion-hardened surface of the orthopedic implant provides a strengthened cam and reduction in wear in the central post of the polymeric tibial insert.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

1. Field of the Invention

The field of this invention relates generally to orthopedic prosthetic devices, and more particularly to posterior stabilized knee prosthetics employing diffusion-hardened surfaces. The invention relates to a knee implant with a diffusion-hardened surface on non-load bearing, non-joint surfaces of the implant for interaction with a polymeric, bio-compatible material, such as UHMWPE.

2. General Background of the Invention

U.S. Pat. No. 5,037,438 and U.S. Pat. No. 5,180,394 to Davidson (which are incorporated by reference) recognized that a thin coating of zirconium oxide, nitride, carbide or carbonitride is especially useful on the portions of prosthetics, especially metallic orthopedic implants for load bearing surfaces which are subject to high rates of wear. An example cited is a femoral head of a hip-system prosthesis which engages a counter-bearing surface in an acetabular cup which is often made of a softer material such as ultra-high molecular weight polyethylene. The Davidson '438 and '394 patents further recognized that zirconium oxide and nitride coatings on non-load bearing surfaces of an orthopedic implant that contact tissue provides a barrier between the metallic prosthesis and body tissue which prevents the release of metal ions and corrosion of the implant.

The zirconium oxide or nitride coating provides the prosthesis with a thin, dense, low friction, wear resistant, bio-compatible surface ideally suited for use on articulating surfaces of joint prostheses wherein a surface or surfaces of the joint articulates, translates or rotates against mating joint surfaces. The zirconium oxide or nitride may be employed on the articulating surfaces of femoral and tibial (miniscal bearing) surfaces of knee joints.

Another Davidson patent, U.S. Pat. No. 5,415,704, (which is incorporated herein by reference) further discusses the creation of a diffusion-hardened surface of bio-compatible metallic metals and alloys, suitable for use as material for a medical implant, including in particular, niobium, titanium, and zirconium based alloys. The '704 patent discusses various methods of oxidizing or nitriding metals and alloys to provide a fine oxide or nitride dispersion.

The Davidson patents, however, did not address the issue of a knee prosthetic having a diffusion-hardened surface, such as a zirconium oxide surface, for non-loading bearing surfaces of the prosthetic that contacts non-load bearing surfaces of a second prosthetic. The Davidson patents only addressed load-bearing articulating joint surfaces having a zirconium oxide surface where the load bearing joint surface either articulated against body tissue or against another load bearing joint surface.

It is known that a common wear problem for a posterior stabilized knee prosthetic exists at the femoral cam-tibial post. Generally, the posterior stabilized knee prosthetic utilizes a cam on a femoral component and a central post on a polymeric tibial insert for stabilization of the knee during flexion motion. During articulation, the polymeric central post contacts the cam of the femoral component. The zones of contact of the femoral cam and the tibial post are both non-load bearing surfaces, however, it has been found that the articulation of the knee prosthetic causes adhesive and abrasive wear to the central post. The wear placed upon the central post generates unwanted polyethylene debris. In cases of a constrained-post prosthetic design, medial and lateral post wear is usually higher, because of resistance of varus-valgus deformation and wear cause by tibial rotation.

Further, the quick flexion motion of the knee will cause the post of the tibial insert to abruptly contact the horizontal cam. In addition, to wear placed upon the central post as discussed above, repeated stressful contact from the central post to the horizontal cam may cause undue cam fatigue ultimately leading to cam deformation or failure.

Therefore, a need exists for a prosthetic implant that provides a strengthened, low friction, highly wear resistant surface on non-load bearing surfaces of the implant where contact occurs with another non-load bearing surfaces a second prosthetic portion. Further, it is desirable that the cam of a posterior stabilized knee prosthetic employ a diffusion-hardened surface to provide reduced wear of the central post improved strength to the central cam.

### SUMMARY OF THE INVENTION

The invention provides a novel prosthetic implant that provides a strengthened, low friction, highly wear resistant surface on non-load bearing surfaces of the prosthetic device where contact occurs with another non-load bearing surface of a second prosthetic device. The contact zones of the non-load bearing surface, although not under the high stress levels and wear rate of load bearing to load bearing surfaces, benefit by the employment of a diffusion-hardened, coated surface on the non-load bearing surface of the prosthetic that contacts the second prosthetic device.

In one embodiment of the invention, the prosthetic implant includes one or more load bearing surfaces and one or more non-load bearing surfaces. The load bearing surfaces of the implant are sized and shaped to engage or articulate with the load bearing surfaces of the second prosthetic device. The second prosthetic device is formed from a bio-compatible, organic polymer or polymer-based composite, such as UHMWPE. A diffusion-hardened surface is employed on the load bearing surfaces and the non-load bearing surface of the prosthetic implant.

Further, the invention is directed to a posterior stabilized knee prosthetic employing a diffusion-hardened surface on non-load bearing surfaces that contact non-load bearing surfaces of a non-load bearing surface of a second prosthetic device. The posterior stabilized knee prosthetic is designed with two condylar portions with a cam extending between the posterior of the condylar portions. The condylar portions are shaped to engage or articulate against a second prosthetic device, namely a tibial insert. The tibial insert is generally made from a bio-compatible, organic polymer or polymer-based composite, such as UHMPE. The tibial insert has a central post that engages with the cam to provide posterior stabilization. A central hole is provided allowing for the central post pass during articulation of the knee. The posterior stabilized knee may be of a constrained design. With this design, the central post a enclosure such as constrained box limits the movement of the central cam.

Additionally, an embodiment of the present invention is a posterior stabilized knee prosthetic system utilizing a femoral component, a tibial insert, and a tibial component. The femoral component is the same prosthetic as discussed in the previous paragraphs. The tibial insert, as discussed above, is shaped to articulate against the femoral component. The tibial insert is designed to fit against the tibial component. The tibial component is designed for surgical implantation into a patient's tibia.

In the embodiments of the invention, the non-load bearing surfaces of the femoral component employ a diffusion-hardened surface where interaction occurs with the non-load bearing surfaces of second prosthetic device. The diffusion-hardened surface provides an improved strengthened, low friction, highly wear resistant surface. Employing a diffusion-hardened surface on the non-load bearing surfaces, e.g. the cam, of the femoral posterior stabilized knee component aids in reducing the problem of wear of the central post of the posterior stabilized knee system.

In one embodiment, the posterior stabilized knee implant is formed from zirconium or a zirconium-based alloy. A diffusion-hardened surface of a thin coating of blue-black or black zirconium oxide is formed on the cam and the condylar portions of the implant. The formation of the diffusion-hardened surface is more generally discussed in the Davidson patents. Some of the metals which may provide a diffusion-hardened surface include one or more of the following metals: hafnium, zirconium, niobium and tantalum. During fabrication of the implant, the thickness of the coating of the diffusion-hardened surface of the cam may vary from the thickness of the coating of the diffusion-hardened surface of the load bearing surfaces.

### BRIEF DESCRIPTION OF THE DRAWINGS

A better understanding of the invention can be obtained from the detailed description of exemplary embodiments set forth below, when considered in conjunction with the appended drawings, in which:

FIG. 1 is a schematic diagram of a regular knee-joint prosthesis;

FIG. 2 is a schematic diagram of FIG. 1 illustrating the regular knee-joint prosthesis implanted in the leg;

FIG. 3 is a shows an embodiment of the present invention as a posterior stabilized knee system;

FIG. 4 is a schematic diagram of the prosthetic knee system of FIG. 3, shown implanted in the femur and tibia, the knee in an extended position; and

FIG. 5 is a schematic diagram of the prosthetic knee system of FIG. 3, show implanted in the femur and tibia, the knee in a flexed position.

### DETAILED DESCRIPTION OF EXEMPLARY EMBODIMENTS

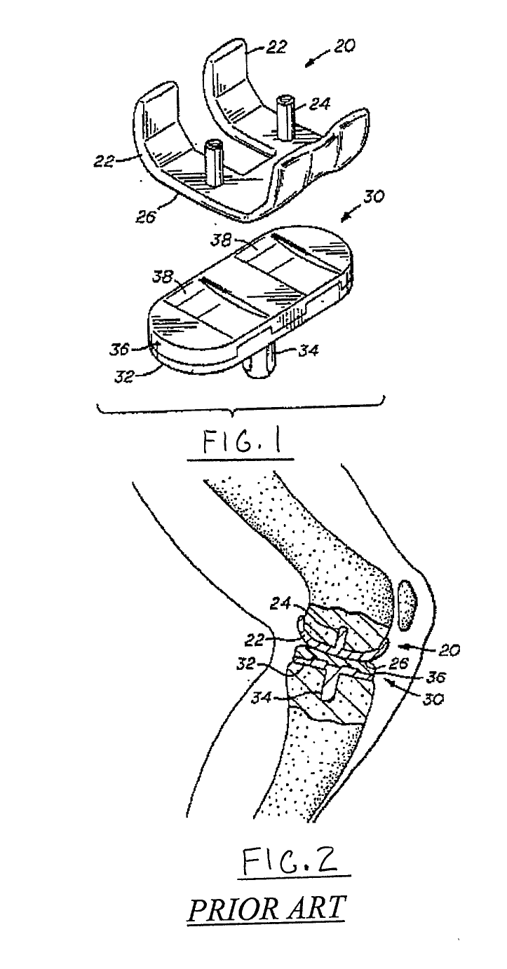

FIGS. 1 and 2 show a typical knee joint prosthesis as disclosed in the prior art where porous bead or wire mesh zirconium oxide coatings can be applied to the tibial or femoral components of the knee or both. The porous metal bead or wire mesh coating is incorporated to allow stabilization of the implant by in-growth of surrounding tissue into the porous coating. The knee joint includes a femoral component 20 and a tibial component 30 with a tibial insert 36. The femoral component includes condyles 22 which provide the articulating surface of the femoral component and pegs 24 for affixing the femoral component to the femur. The tibial component 30 includes a tibial base 32 with a peg 34 for mounting the tibial base onto the tibia. A tibial platform 36 is mounted atop the tibial base 32 and is supplied with grooves 38 similar to the shape of the condyles 22. The bottom surfaces of the condyles 26 contact the tibial platform's grooves 38 so that the condyles articulate within these grooves against the tibial platform. While condyles are typically fabricated of metals, the tibial platform may be made from an organic polymer or a polymer-based composite. The hard metallic condyle surfaces 26 articulate against a relatively softer organic composition. Zirconium oxide or nitride may be employed on the condyles for articulation with the load-bearing surfaces tibial grooves 38.

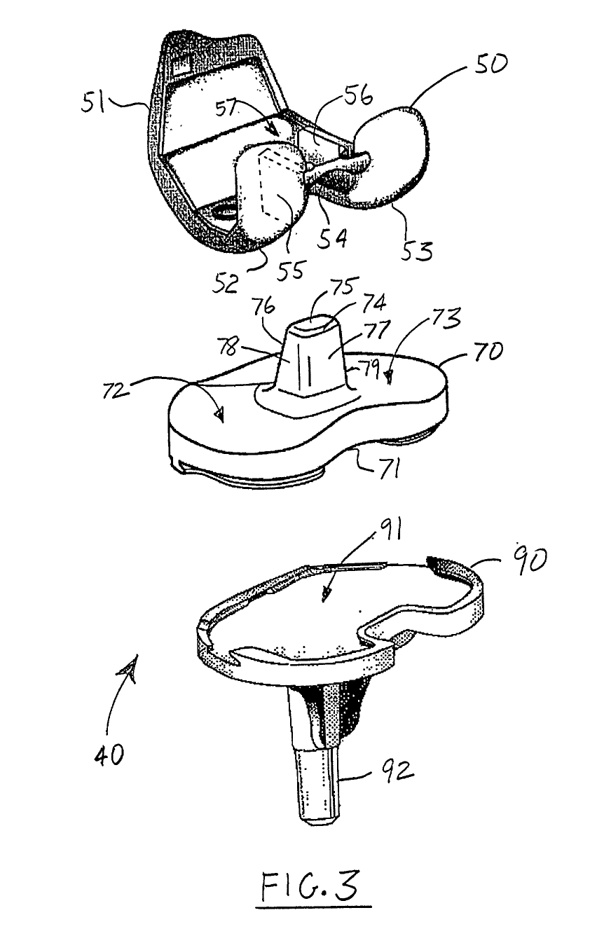

FIG. 3 shows an embodiment of the present invention designated generally by the numeral 40. In FIG. 3, a posterior stabilized knee prosthetic system is shown including a femoral component 50, polymeric articulating insert 70 and tibial component 90. When implanted, the femoral component 50 articulates during flexion motion against the polymeric insert 70. The polymeric insert 70 is mated with the tibial component 90.

The femoral component 50 has anterior portion 51 and a pair of condylar portions 52. Femoral component 50 has central opening and a horizontal bar cam 54 that extends between the posterior of the condylar portions 52, 53. A pair of vertical walls 55, 56 extend along opposing sides of central opening and connect to both of the posterior condylar portions 52, 53 and to horizontal bar 54. The vertical walls 55, 56 can be generally parallel. Another embodiment of the femoral component, is a constrained design where the vertical walls are connected with a distal wall which is connected with the cam and an anterior wall. In the constrained design, the cam may be integrally formed with the distal wall. Instead of an open hole through which the post may travel, the constrained design forms an enclosure with a cam between the condylar portions thereby limiting the articulation of the femoral component. The enclosure usually is formed as a three-sided box between the condylar portions with the cam integrally molded with the proximal wall.

The horizontal cam employs a diffusion-hardened surface. In one embodiment, a zirconium oxide coating is formed on the cam though oxidation of a zirconium or zirconium-based alloy from which the femoral component is made. After the oxide coating on the cam is formed, the oxide coating may be polished to exhibit a mirror-like finish. The cam's diffusion-hardened surface results in added strength to the cam. Additionally, reduction of wear to the central post will be achieved over other metals, such as cobalt chrome, that are utilized for the manufacture of a knee prosthetic.

In a constrained design, a zirconium oxide coating is formed on the inside of the constrained-box and cam where the central post 74 of the polymeric insert 70 interacts with the inner walls of the constrained-box. The oxide coating of the inner walls and cam of the constrained box may also be polished to exhibit a mirror-like finish.

In addition, to the oxide coating being formed on the cam and inner walls of the constrained-box, a diffusion-hardened surface is preferably formed on the condylar portions of the femoral component. For example, the formation of a coating of oxidized zirconium provides reduced wear to the load bearing condylar portions and the pair of concavities 72, 73 of the polymeric insert 70.

The polymeric insert 70 has a generally planar distal surface 71 and a proximal surface with a pair of concavities 72, 73. In the embodiment of FIG. 3, insert 70 has a central post 74. The central post has a proximal surface 75, anterior surface 76, posterior surface 77, and sides 78, 79. Although the central post is generally rectangular the post make be shaped differently.

The polymeric insert 70 is affixed or fitted to a tibial component 90, commonly referred to as a tibial tray. The tibial component has a proximal surface 91 where the polymeric insert 70 engages with the tibial component 90. The tibial component has a stem 92 for implantation to the patient's tibia.

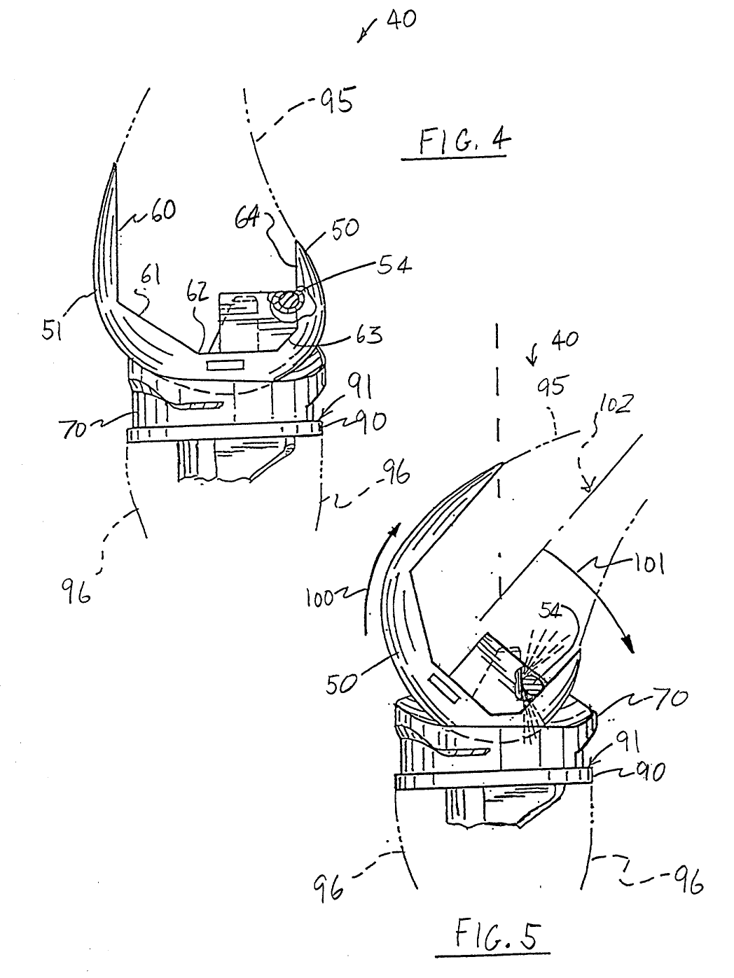

Referring now to FIG. 4, the prosthetic knee system is shown implanted in the femur 95 and tibia 96. Femoral component 50 is shown in a longitudinal resting position on the polymeric insert 70 which is matted with the tibial component 90. Femoral component 50 provides a plurality of flat surfaces that register against and conform to surgically cut flat surfaces that are provided on the patient's distal femur 95. These flat surfaces include flat surface 60 is an anterior surface, surface 61 which is a diagonally extending anterior surface that spans between anterior surface 60 and distal surface 62. Distal surface 62 spans between diagonal surface 61 and posterior diagonal surface 63. Posterior surface 64 is generally parallel to anterior flat surface 60. These five flat surfaces 60-64 of the femoral component 50 register against and conform to five surgically cut surfaces on a patient's distal femur 95. Femoral component 50 can be securely fashioned to the patient's distal femur 95 using bone cement for example.

As is illustrated in FIG. 5, a range of motion for the patient's knee fitted with the knee prosthesis 40 as illustrated with arrows 100, 101. For purposes of reference, the patient's central longitudinal axis 102 of the distal femur 95 is shown rotating in the direction of arrow 100. In the flexed position shown, the horizontal bar cam 54 of femoral component 50 registers against the posterior surface 77 of central post 74 of polymeric insert 70. In this position, the central post 74 causes femoral roll back on the tibia articular insert 70. The posterior aspect of the tibia articular surface at 77 provides a lift that is created by generally following the curvature of the femoral component 50 in extension. This will provide a high degree of surface contact, conformity, subsequently providing low contact stress, in extension, where most of gait occurs. The post 74 can have a square or rectangular base that fits snugly with the central opening 57 of the femoral component 50.

By providing the posterior stabilized design with the central post 74, as the knee is flexed, the horizontal bar cam 54 acts as a cam on the femoral component 50 to engage the post 70 at surface 77 on the polymeric insert 70, causing the femoral posterior condyles 52,53 to roll back onto the tibial articular concavity surfaces 72, 72.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the invention described in the specification. As one of ordinary skill in the art will readily appreciate from the disclosure of the present invention, devices, means, metals and alloys existing or later to be developed that perform substantially the same function or achieve substantially the same result as the corresponding embodiments described herein may be utilized according to the present invention. Accordingly, the appended claims are intended to include within their scope such devices, means, and metals and alloys.

What is claimed is:

1. A posterior stabilized knee prosthetic system comprising:
a) a femoral component configured to be surgically implanted into a patient's femur, the femoral component having two condylar portions with a cam extending between the posterior end of the condylar portions, and the cam having a diffusion-hardened surface along a portion of the cam for adding strength and wear resistance to the contact zones of the cam;
b) a tibial component configured to be surgically implanted into a patient's tibia; and
c) a tibial insert having a proximal surface that is shaped to articulate against the femoral component, the insert having a distal surface that fits against the proximal surface of the tibial component, and the tibial component having a post for engaging the femoral component to provide posterior stabilization.

2. The prosthetic system of claim 1, wherein the diffusion-hardened surface is a thin coating of blue-black or black zirconium oxide.

3. The prosthetic system of claim 1, wherein the diffusion-hardened surface is a thin coating of oxidized metal selected from one or more metals from the group consisting of hafnium, zirconium, niobium and tantalum.

4. The prosthetic system of claim 2, wherein the condylar portions have a load bearing surface with a thin coating of a blue-black or black zirconium oxide.

5. The prosthetic system of claim 4, wherein the thickness of the zirconium oxide is greater than the thickness of the thickness of the zirconium oxide of the cam.

6. The prosthetic system of claim 3, wherein the femoral component has a pair of generally parallel vertical walls connected to the inner sides of the posterior condylar portions, wherein the vertical walls have an inner-side with a diffusion-hardened surface, wherein the diffusion-hardened surface is a thin coating of oxidized metal selected from one or more metals from the group consisting of hafnium, zirconium, niobium and tantalum.

7. The prosthetic system of claim 6, wherein the femoral component has a constrained box formed by an anterior wall connected to the pair of vertical walls, and a proximal wall connected to the pair of vertical walls, wherein the anterior wall is connected to or integrally formed with the cam, wherein the inner-side of the proximal anterior walls have a thin coating of oxidized metal selected from one or more metals from the group consisiting of hafnium, zirconium, niobium, and tantalum.

8. The prosthetic system of claim 3, wherein the tibial component is made from a polymeric bio-compatible material.

9. The prosthetic system of claim 8, wherein the polymeric bio-compatible is UHMWPE.

10. A prosthesis for implantation in a patient, comprising:
a prosthesis body for implantation in the body, the prosthesis body having one or more load bearing surfaces and one or more non-load bearing surfaces,
the load bearing surface on the prosthesis body being sized and shaped to engage or cooperate with a second load bearing surface on another prosthesis portion, said second load bearing surface being formed of an organic polymer or polymer-based composite,
the non-load bearing surface on the prosthesis body being sized and shaped to engage or cooperate with a second non-load bearing surface on another prosthesis portion, said second non-load bearing surface being formed of an organic polymer or polymer-based composite,
a diffusion-hardened coated surface on the bearing surface, and
a diffusion-hardened coated surface on the non-load bearing surface.

11. The prosthesis of claim 10, wherein the diffusion-hardened surface is a thin coating of blue-black or black zirconium oxide.

12. The prosthesis of claim 10, wherein the diffusion-hardened surface is a thin coating of oxidized metal selected from one or more metals from the group consisting of hafnium, zirconium, niobium and tantalum.

13. The prosthesis of claim 12, wherein the thickness of the coating of the diffusion-hardened surface of the load bearing surface is greater than the coating of the diffusion-hardened surface of the non-load bearing surface

14. The prosthesis of claim 10, wherein the prosthesis body has two condylar portions with a cam extending between the posterior of the condylar portions, the condylar portions having a load-bearing surface and the cam having a non-load bearing surface.

15. The prosthesis of claim 14, wherein the condylar portions are shaped to articulate against a tibial insert having a post for engaging the prosthesis body to provide posterior stabilization.

16. A posterior stabilized knee prosthetic comprising:
a femoral component configured to be surgically implanted into a patient's femur, the femoral component having two condylar portions with a cam extending between the posterior of the condylar portions, the cam having a having diffusion-hardened surface along a portion of its length for adding strength to the impact zones of the cam;
wherein the condylar portions are shaped to articulate against a tibial insert having a post for engaging the femoral component to provide posterior stabilization.

17. The posterior stabilized knee prosthetic of claim 16, wherein the diffusion-hardened surface is a thin coating of oxidized metal selected from one or more metals from the group consisting of hafnium, zirconium, niobium and tantalum.

18. The posterior stabilized knee prosthetic of claim 17, wherein the cam is shaped as a horizontal bar allowing femoral component rollback on the central post of the tibial insert.

19. The posterior stabilized knee prosthetic of claim 17, wherein the condylar portions have a load bearing surface with a thin coating of a blue-black or black zirconium oxide.

20. The prosthetic stabilized knee prosthetic of claim 17, wherein the femoral component has a constrained-box integrally formed with the cam, the constrained-box being connected to the posterior of the condylar portions, wherein the inner-sides of the constrained-box have a thin coating of oxidized metal selected from one or more metals from the group consisting of hafnium, zirconium, niobium and tantalum.
